(19) United States
(12) Patent Application Publication
(10) Pub. No.: US 2013/0253555 A1
(43) Pub. Date: Sep. 26, 2013

(54) OPHTHALMIC SURGICAL METHOD COMPRISING A STEP OF AT LEAST PARTLY REMOVING THE VITREOUS HUMOUR

(71) Applicant: BBS S.R.L., Padova (IT)

(72) Inventors: Mauro BECCARO, Cadoneghe (IT); Enrico BETTINI, Fiesso D'artico (IT); Paolo SIGNORI, Verona (IT)

(73) Assignee: BBS S.R.L., Padova (IT)

(21) Appl. No.: 13/663,870

(22) Filed: Oct. 30, 2012

(30) Foreign Application Priority Data
Mar. 20, 2012 (IT) VR2012A000051

(51) Int. Cl. A61B 17/3205

(52) U.S. Cl. 606/167

(57) ABSTRACT

An ophthalmic surgical method comprising a step of at least partly removing the vitreous humour, which also comprises a step of highlighting the vitreous to be removed using a coloured composition which selectively highlights the vitreous, allowing a distinction between the vitreous and the other structures of the eye, said distinction facilitating vitreous removal, wherein the coloured composition comprises a polymeric dye comprising at least one backbone to which at least one reactive dye is chemically linked; in a preferred embodiment the polymeric dye being a blue dextran.

This invention relates to an ophthalmic surgical method comprising a step of at least partly removing the vitreous humour.

In particular, this invention is primarily intended for all vitreoretinal surgical methods involving a step of removing the vitreous humour (vitrectomy) from the back or vitreous chamber, that is to say, the space between the rear surface of the crystalline lens and the retina.

At present, when it is necessary to remove the vitreous humour (hereinafter referred to simply as the vitreous), usually three holes are made in the sclera to give access to the vitreous chamber. A saline solution irrigation device is connected to a first hole, while a suction device is connected to a second hole. Keeping both the irrigation and suction devices operative, the surgeon then inserts a vitrectomy instrument in the vitreous chamber and uses the instrument to cut up the vitreous into fragments. The small pieces obtained can be sucked out both through the suction device and through the vitrectomy instrument if the latter is equipped with a further suction unit.

Elimination of the vitreous, a transparent, colourless polysaccharide-based gel which fills the vitreous chamber, is relatively simple as long as one is dealing with the part of the vitreous located at the centre of the vitreous chamber. In fact, the surgeon simply has to gradually move the tip of the vitrectomy instrument around the entire central space of the vitreous chamber.

In contrast, the situation becomes more complicated regarding the parts of the vitreous which are located against the walls delimiting the vitreous chamber, that is to say, the retina and the crystalline lens. In fact, the surgeon must absolutely avoid touching the walls with the vitrectomy instrument, or the patient could be left with injuries.

Moreover, since the vitreous is a transparent, colourless substance, it is difficult to ascertain whether or not there are residues dose to the was of the vitreous chamber. Therefore, the surgeon must rely solely on his expertise in recognising the different reflective capacity of the vitreous compared with the lavage fluid.

To attempt to overcome that problem, in recent years efforts were made to render the vitreous more visible using some type of highlighter/colouring.

In particular, for that purpose use spread of a suspension based on micronized triamcinolone acetonide, a substance already known as a cortisonic drug which has a white colour. Said substance is used in micronized powder form, in an emulsion which can be injected into the vitreous chamber (see, for example, U.S. Pat. No. 6,395,294). Once injected into the eye the micronized triamcinolone acetonide, which is an insoluble powder, tends to adhere to the vitreous, making it visible by means of a kind of coating.

However, said solution is not without disadvantages.

First, triamcinolone acetonide is a non-transparent, opaque powder which once deposited on a structure obstructs the view of everything behind it. Consequently, it must be injected into the vitreous in two or more injections one after another to gradually highlight only small portions of the vitreous.

The second problem is due to the fact that the triamcinolone acetonide adheres not just to the vitreous, but also to the walls of the vitreous chamber. Consequently, when dealing with the final vitreous residues, the possibility of effectively removing them is again left to the skill of the surgeon.

It should also be noticed that the particle size measurement of the powder may cause cases of tissue inflammation, since the body may perceive it to be a foreign body.

Similar considerations apply for other compositions consisting of emulsions or suspensions, such as those described in patent applications US 2007/0225727, US 2008/0147039 and WO 2005/072744.

As an alternative to the use of substances able to highlight the vitreous by a physical effect of coating it with micronized powders, over the years some attempts have been made to perfect coloured solutions, such as those described in patent applications US 2003/0096334, ON 101125149, JP 2007191454 and WO20061133903 and in patents RU 2371151 and RU 2193376.

However, so far, none of the proposed solutions has been able to guarantee selective colouring with sufficient duration of the vitreous using physiologically compatible substances.

In fact, many dyes tend to spread in the vitreous chamber and to also colour other structures of the eye.

Second, this invention is generally intended for all ophthalmic surgical methods in which it is necessary to at least partly remove the vitreous and in which it is necessary or at least useful to highlight the vitreous.

In particular, problems similar to those described above may also be encountered as regards work in the anterior chamber, above all during cataract surgery. During the work, it may happen (due either to physiological problems with the eye, or to human error) that a certain quantity of vitreous is transferred into the anterior chamber, for example following localised piercing of the rear capsule of the crystalline lens. In most cases the transfer occurs within the capsule of the crystalline lens after the lens has been removed.

The presence of vitreous in the anterior chamber on one hand may give rise to inflammations, and on the other hand, if it affects the capsule during cataract surgery, it may prevent correct positioning of the artificial lens (IOL).

Consequently, every time there is a transfer of vitreous into the anterior chamber, the surgeon must remove it. However, at that point, the surgeon is faced with all of the problems involved in seeing the vitreous which are described above with reference to vitreoretinal surgery.

In this context, the technical purpose which forms the basis of this invention is to provide an ophthalmic surgical method comprising a step of at least partly removing the vitreous, which overcomes the above-mentioned disadvantages.

In particular, the technical purpose of this invention is to provide an ophthalmic surgical method comprising a step of at least partly removing the vitreous, which guarantees selective highlighting of the vitreous in a physiologically compatible way.

The technical purpose specified and the aims indicated are substantially achieved by an ophthalmic surgical method comprising a step of at least partly removing the vitreous as described in the appended claims.

Further features and the advantages of this invention are more apparent in the detailed description of several preferred, non-limiting embodiments of an ophthalmic surgical method comprising a step of at least partly removing the vitreous according to this invention.

In accordance with this invention, the ophthalmic surgical method, comprising a step of at least partly removing the vitreous, also comprises a step of highlighting the vitreous to be removed by using a coloured composition. In fact, the latter selectively highlights the vitreous, creating a clear distinction between the vitreous and the other structures of the eye, a distinction which therefore facilitates removal of the vitreous. Said distinction during the work facilitates complete removal of the relevant vitreous (all or part of it), since the vitreous is a different colour to the other structures of the eye (although remaining substantially transparent, thus allowing the surgeon to see the structures of the eye which are behind the vitreous). As described in more detail below, the coloured composition comprises a polymeric dye comprising at least one backbone to which at least one reactive dye is chemically linked.

In particular, according to a first embodiment of this invention, the surgical method is a vitreoretinal surgical method and comprises a step of completely removing the vitreous. In that case, the coloured composition is usually injected into the vitreous chamber where the polymeric dye is rapidly inserted in the vitreous. In this way the vitreous visibly takes on the colour of the coloured composition and is highlighted while remaining substantially transparent. To highlight the vitreous more rapidly, the coloured solution is preferably injected into the vitreous chamber in an off-centre position, maintaining active the irrigation of the vitreous chamber with a saline solution or the like.

Even more preferably, the injection is performed in a peripheral position and in favour of the irrigation flow. This allows a very fast distribution of the coloured composition throughout the vitreous.

In addition, the coloured composition can also be used in the eye after the injection of perfluorinated liquid used for retinal tamponade. In fact, in this case the coloured composition floats on the perfluorinated liquid and can easily make contact with the rear capsule of the crystalline lens to highlight any residues of vitreous still attached to it. The surgeon can then see any vitreous by pressing the eyeball inwards with one finger.

In contrast, according to a second embodiment of this invention, the surgical method comprises work in the anterior chamber of the eye or on the crystalline lens, and the coloured composition is used to selectively highlight any vitreous which has come out of the vitreous chamber.

In this case, application of the coloured composition may preferably be by means of simple injection into the anterior chamber (or in particular into the capsule) with subsequent rinsing away of the excess composition, or by direct application on the vitreous to be removed.

The effective injection of the coloured composition into the eye may usually be performed in two ways: either by injecting the coloured composition directly into the eye, or inserting it in the irrigation fluid. In the former the injection is through a hole in the eye (only hole or trocar), different to that connected to the infusion/irrigation pipe, in which the dedicated syringe and cannula are inserted. In contrast, in the latter case a four-way valve (or a two-way valve connected in series to a three-way valve) is fitted in the infusion pipe, outside the eye and in particular upstream of the pipe leading to the tube (approx. 30 cm). It should be noticed that at present along said pipe a three-way valve is already fitted, which is designed, by closing and opening, to allow the selective supply of air or liquid into the eye. Consequently, when implementing this invention it is possible to either add a two-way valve in series with the three-way valve, or to substitute the latter with a four-way valve. In both cases, using the additional way it is possible to directly inject the dye into the irrigation flow without inserting a dedicated syringe and cannula in the eye.

As already indicated, according to this invention, the coloured composition used in the surgical method comprises at least one polymeric dye comprising at least one backbone to which at least one reactive dye is chemically linked.

It should be noticed that in the context of this invention the term “backbone” refers to the main molecule to which one or more molecules of a reactive dye are chemically linked. The term “reactive dye” refers to any dye containing reactive groups.

Advantageously, the backbone comprises a polysaccharide or a protein or a peptide or a derivative of them. Even more advantageously, the backbone comprises dextran.

Regarding the reactive dye, amongst the various possible dyes those known with the brand names Cibacron®, Procion®, Reacton® and Remazol® can be mentioned.

In particular, it is particularly advantageous to use as the reactive dye a reactive blue, such as reactive blue 2 (also known as Cibacron F3GA). As is known, the latter has the following structure:

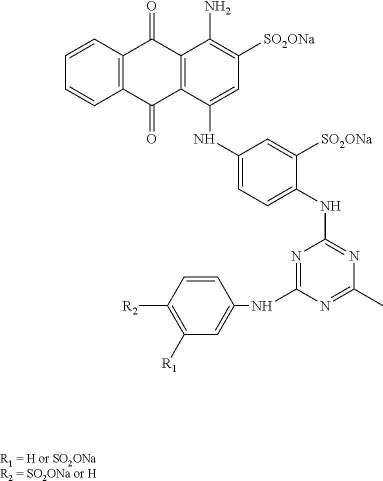

In the most preferred embodiment, the polymeric dye is a blue dextran (CAS 87915-38-6), such as that marketed by Sigma-Aldrich of Saint Louis, Mo., USA.

However, in general the polymeric dye is soluble in water and, advantageously, also in buffered solutions produced with monovalent and bivalent ions.

Advantageously, both generally and in the particular case of blue dextran, the polymeric dye has a molecular weight of between 4,000 and 500,000,000 Daltons, preferably being between 40,000 and 2,000,00( ) Daltons.

In the preferred embodiments, the coloured composition is a solution or a mixture of the polymeric dye in a base liquid such as a PBS (phosphate buffered saline) water-based solution or a generic saline solution (for example sodium chloride-based).

Advantageously, at least at the moment of use, the polymeric dye is present in the composition in a quantity by weight of between 0.1% and 1%, and even more preferably between 0.3% and 0.8% and between 0.4% and 0.6%. However, depending on requirements, the composition may even have a polymeric dye concentration which is greater and it must be diluted only at the moment of use.

Preferably, the coloured composition has a pH of between 6.5 and 7.5.

Moreover, at least in a temperature range of between 10° C. and 50° C., the coloured composition is a liquid.

Finally, it should be noticed that the colouring intensity of the coloured composition must be such that, once it has been inserted in the vitreous, the latter is highlighted by the colouring, but remains substantially transparent. That means that the coloured composition must allow what is behind the vitreous to be seen, In particular, when used in the ophthalmic surgical method, it must allow the structures of the eye, such as the retina, behind the vitreous to be seen. Moreover, in the temperature range indicated the viscosity of the coloured composition must be such that it can be injected into the eye using a normal syringe.

This invention brings important advantages.

This invention allows more precise removal of the vitreous thanks to the fact that it is selectively highlighted without changing the colour of the surrounding structures of the eye, therefore making the vitreous visible.

The coloured solution used is also physiologically compatible.

Moreover, tests run by the Applicant, although having shown a tendency of the coloured composition to be washed away following irrigation of the eye and aspiration during the vitrectomy, demonstrated that the time required to wash it away is long enough to allow for example an entire vitrectomy to be carried out at most with just a few injections of coloured composition in the vitreous chamber (even just one, depending on the speed of the surgeon performing the vitrectomy). It should also be noticed that the fact that the highlighting provided by the coloured composition is not permanent is advantageous because it ensures that by the end of the surgery any residues of coloured composition not identified by the surgeon have also been removed.

A further advantage of the coloured composition used in the context of this invention is the fact that it cannot colour the perfluorinated liquids which are usually inserted in the eye as a retinal tamponade, after removal of the vitreous. In fact, in this way, it is easy to identify any vitreous residues floating on the perfluorinated liquid (due to the lower density of the vitreous).

A further advantage linked in particular to the preferred embodiment comprising the use of blue dextran as the polymeric dye is the fact that said substance is very heat stable, a property which allows it to be steam sterilised without the risk of compromising its integrity.

Finally, it should be noticed that this invention is relatively easy to produce and that even the cost linked to implementing the invention is not very high.

The invention described above may be modified and adapted in several ways without thereby departing from the scope of the inventive concept.

Moreover, all details of the invention may be substituted with other technically equivalent elements and the materials used, as well as the shapes and dimensions of the various components, may vary according to requirements.

1. An ophthalmic surgical method comprising a step of at least partly removing the vitreous humour, also comprising a step of highlighting the vitreous to be removed using a coloured composition which selectively highlights the vitreous, allowing a distinction between the vitreous and the other structures of the eye, said distinction facilitating vitreous removal, wherein the coloured composition comprises a polymeric dye comprising at least one backbone to which at least one reactive dye is chemically linked.

2. The method according to the preceding claim, wherein the surgical method is a vitreoretinal surgical method and comprises a step of completely removing the vitreous.

3. The method according to the preceding claim, wherein the coloured solution is injected into the vitreous chamber.

4. The method according to claim 3, wherein, the coloured solution is injected into the vitreous chamber in an off-centre position, maintaining active irrigation of the vitreous chamber with a saline solution or the like.

5. The method according to claim 4, wherein the coloured solution is injected into the vitreous chamber in favour of the irrigation flow.

6. The method according to claim 1 comprising surgical work in the anterior chamber of the eye or on the crystalline lens, and in which the coloured composition is used to selectively highlight any vitreous which has come out of the vitreous chamber.

7. The method according to claim 1, wherein the polymeric dye is soluble in water and/or in buffered solutions produced with monovalent and bivalent ions.

8. The method according to claim 1, wherein the backbone comprises a polysaccharide or a protein or a peptide or a derivative of them, and/or wherein the reactive dye is a reactive blue, in particular reactive blue 2 or a blue dextran.

9. The method according to claim 1, wherein the polymeric dye has a molecular weight of between 4,000 and 500,000,000 Daltons, preferably being between 40,000 and 2,000,000 Daltons.

10. The method according to claim 1, wherein the polymeric dye is present in the composition in a quantity by weight of between 0.1% and 1%, or between 0.3% and 0.8% or between 0.4% and 0.6%.

11. The method according to claim 1, wherein the polymeric dye is dissolved in, or mixed with, a PBS or saline solution.

12. The method according to claim 1, wherein the coloured composition has a pH of between 6.5 and 7.5.

13. The method according to claim 3, wherein the polymeric dye is soluble in water and/or in buffered solutions produced with monovalent and bivalent ions.

14. The method according to claim wherein the backbone comprises a polysaccharide or a protein or a derivative of them, and/or wherein the reactive dye is a reactive blue, in particular reactive blue 2 or a blue dextran.

15. The method according to claim 3, wherein the polymeric dye has a molecular weight of between 4,000 and 500,000,000 Daltons, preferably being between 40,000 and 2,000,000 Daltons.

16. The method according to claim 3, wherein the polymeric dye is present in the composition in a quantity by weight of between 0.1% and 1%, or between 0.3% and 0.8% or between 0.4% and 0.6%.

17. The method according to claim 3, wherein the polymeric dye is dissolved in, or mixed with, a PBS or saline solution.

18. The method according to claim 3, wherein the coloured composition has a pH of between 6.5 and 7.5.

19. The method according to claim 5, wherein the polymeric dye is soluble in water and/or in buffered solutions produced with monovalent and bivalent ions.

20. The method according to claim 5, wherein the backbone comprises a polysaccharide or a protein or a derivative of them, and/or wherein the reactive dye is a reactive blue, in particular reactive blue 2 or a blue dextran.

21. The method according to claim 5, wherein the polymeric dye has a molecular weight of between 4,000 and 500,000,000 Daltons, preferably being between 40,000 and 2,000,000 Daltons,

22. The method according to claim 5, wherein the polymeric dye is present in the composition in a quantity by weight of between 0.1% and 1%, or between 0.3% and 0.8% or between 0.4% and 0.6%.

23. The method according to claim 5, wherein the polymeric dye is dissolved in, or mixed with, a PBS or saline solution.

24. The method according to claim 5, wherein the coloured composition has a pH of between 6.5 and 7.5.

25. The method according to claim 6, wherein the polymeric dye is soluble in water and/or in buffered solutions produced with monovalent and bivalent ions.

26. The method according to claim 6, wherein the backbone comprises a polysaccharide or a protein or a derivative of them, and/or wherein the reactive dye is a reactive blue, in particular reactive blue 2 or a blue dextran.

27. The method according to claim 6, wherein the polymeric dye has a molecular weight of between 4,000 and 500,000,000 Daltons, preferably being between 40,000 and 2,000,000 Daltons.

28. The method according to claim 6, wherein the polymeric dye is present in the composition in a quantity by weight of between 0.1% and 1%, or between 0.3% and 0.8% or between 0.4% and 0.6%.

29. The method according to claim 6, wherein the polymeric dye is dissolved in, or mixed with, a PBS or saline solution.

30. The method according to claim 6, wherein the coloured composition has a pH of between 6.5 and 7.5.
